(19) United States
(12) Patent Application Publication
(10) Pub. No.: US 2023/0053429 A1
(43) Pub. Date: Feb. 23, 2023

(54) SYSTEM AND METHOD FOR AUTOMATIC ANALYSIS OF TEXTS IN PSYCHOTHERAPY, COUNSELING, AND OTHER MENTAL HEALTH MANAGEMENT ACTIVITIES

(71) Applicant: Mind Medicine, Inc., New York, NY (US)

(72) Inventors: Adam KOLAR, Vinohradska (CZ); Martin MAJERNIK, Senec (SK); Miguel Amavel dos Santos PINHEIRO, Praha (CZ); Daniel R. KARLIN, New York, NY (US); Robert BARROW, Madison, WI (US)

(73) Assignee: Mind Medicine, Inc., New York, NY (US)

(21) Appl. No.: 17/889,415

(22) Filed: Aug. 17, 2022

Related U.S. Application Data
(60) Provisional application No. 63/234,336, filed on Aug. 18, 2021.

(51) Int. Cl. A61B 5/16; A61B 5/00

(52) U.S. Cl. CPC A61B 5/16; A61B 5/4803

(57) ABSTRACT

A method of analyzing a patient's mental state, by automatically processing, integrating, and analyzing text-based and audio-based sources from the patient and clinical staff, and generating real-time outcomes and predictions. A system for processing, analyzing, and managing a patient's input including a data pool, model HUB, search service, topic modeling service, mental health related prediction service, and analytics all in electronic communication. A method of analyzing a patient, by processing, analyzing, and managing a patient's and clinical staff's text and audio input, and informing and augmenting diagnostic and prognostic processes, identifying improvement and deterioration of a patient's mental state, and identifying adverse events in psychotherapy, counseling, and other mental health management activities. A system for processing, analyzing, and managing clinical and diagnostic texts, and audio transcripts.

### BACKGROUND OF THE INVENTION

### 1. Technical Field

The present invention relates to systems and methods for analyzing the mental state of a patient.

### 2. Background Art

The mental state of a patient can be assessed during a medical appointment by medical personnel. General observations can be made as well as specific tests performed such as for attention, executive functioning, gnosia, language, memory, orientation, praxis, prosody, thought content, thought processes, and visuospatial proficiency.

Currently applied processes and methods in psychotherapy, counseling, and other mental health management activities are generating excessive amounts of text-based and audio-based data inputs from both the patients and the clinical staff.

Data originating from patients usually are in the form of free-text and structured text responses to diagnostic assignments and tasks, patient's diaries, patient journals, worry scripts, transcripts of patient-clinician discussions from the patient's treatment and therapy, written and transcribed answers to a structured set of questions, and audio recordings. Data originating from clinical staff usually are in the form of clinical staff's clinical notes from patient's treatment and therapy, initial assessments notes, progress notes, non-clinical notes from the patient's treatment and therapy, drug administration notes, non-clinical and research staff notes, treatment plans, prescriptions, audio recordings, and release documentation.

While all the above data sources hold immense clinical, diagnostic and prognostic value, it is currently impossible to efficiently process these massive unstructured data sources in a comprehensive, clinically relevant output.

Systems have been developed to assess mental status. For example, U.S. Patent Application Publication No. 20170119297 discloses A computer-implemented method of assessing a mental state of a subject (106) includes receiving (302), as input, a heartbeat record (200) of the subject. The heartbeat record comprises a sequence of heartbeat data samples obtained over a time span which includes a pre-sleep period (208), a sleep period (209) having a sleep onset time (224) and a sleep conclusion time (226), and a post-sleep period (210). At least the sleep onset time and the sleep conclusion time are identified (304) within the heartbeat record. A knowledge base (124) is then accessed (306), which comprises data obtained via expert evaluation of a training set of subjects and which embodies a computational model of a relationship between mental state and heart rate characteristics. Using information in the knowledge base, the computational model is applied (308) to compute at least one metric associated with the mental state of the subject, and to generate an indication of mental state based upon the metric. The indication of mental state is provided (310) as output.

U.S. Patent Application Publication No. 20130297536 discloses a system and method for monitoring a user's mental health and collecting data. The user's use of electronic devices is tracked, such as usage of his mobile phone, tablet and his web activity. The invention “learns” each patient's unique behavioral patterns to be used as a “base line” representing the steady state (chronic phase) of the patient. The algorithmic processing unit detects any irregularities in a patient's behavioral patterns and produces a deterioration prediction. If it is determined that a threshold is exceeded, an alert is sent to a health professional.

There remains a need for a system that can effectively monitor mental health of a patient that can integrate text-based sources of information.

### SUMMARY OF THE INVENTION

The present invention provides for a method of analyzing a patient's mental state, by automatically processing, integrating, and analyzing text-based and audio-based sources from the patient and clinical staff, and generating real-time outcomes and predictions.

The present invention provides for a system for processing, analyzing, and managing a patient's input including a data pool, model HUB, search service, topic modeling service, mental health related prediction service, and analytics all in electronic communication.

The present invention also provides for a method of analyzing a patient, by processing, analyzing, and managing a patient's and clinical staff's text and audio input, and informing and augmenting diagnostic and prognostic processes, identifying improvement and deterioration of a patient's mental state, and identifying adverse events in psychotherapy, counseling, and other mental health management activities.

The present invention also provides for a system for processing, analyzing, and managing clinical and diagnostic texts, and audio transcripts including a data pool, model HUB, search service, topic modeling service, mental health related prediction service, and analytics all in electronic communication, wherein said system is able to identify and highlight recurrent topics, critical moments, and clinically valuable moments in clinical staff's feetext notes, to manage content/information load for clinical professionals, and to offer a retrospective view or an overview of a patient's treatment progress.

The present invention provides for a method of analyzing clinical and diagnostic texts, by processing, analyzing, and managing clinical and diagnostic texts, identifying and highlighting recurrent topics, critical moments, and clinically valuable moments in clinical staff's feetext notes, managing content/information load for clinical professionals, and offering a retrospective view or an overview of a patient's treatment progress.

### DESCRIPTION OF THE DRAWINGS

Other advantages of the present invention are readily appreciated as the same becomes better understood by reference to the following detailed description when considered in connection with the accompanying drawings wherein:

FIG. 1 is a diagram of the system architecture;

FIG. 2 is a diagram of the active learning and data collection system;

FIG. 3 is a diagram of assessment of pathological and non-pathological mental states (classification) from text;

FIG. 4 is a diagram of assessment of specific mental disorder severity and progression from text;

FIG. 5 is a diagram of highlighting parts of text with high mental disorder severity;

FIG. 6 is a diagram of topical analysis of psychotherapeutic texts and patients' reports;

FIG. 7 is a diagram of projecting psychotherapeutic session context into a topic map of relations;

FIG. 8 is a diagram of predicting topics and keywords occurrence in a next session based on their presence (frequency/criticality) in previous sessions;

FIG. 9 is a diagram of a search for patients reporting similar behavioral, psychological, or emotional symptoms, indications, or states;

FIG. 10 is a diagram of summarization of large volume of psychotherapeutic texts into a shorter abstract;

FIG. 11 is a diagram of question answering on the psychotherapeutic text;

FIG. 12 is a diagram of text search over the psychotherapeutic transcript based on text model embeddings, restricted by clinically relevant topics, systems, or indications;

FIG. 13 is a diagram of pairing critical psychological events, with clinical relevance (or topics), with concrete entities in the text identified via an entity recognition model; and

FIG. 14 shows therapy notes.

### DETAILED DESCRIPTION OF THE INVENTION

The present invention provides for a system for processing, analyzing, and managing a patient's input. The purpose of this system is to inform and augment diagnostic and prognostic processes, to identify improvement and deterioration of a patient's mental state, and to identify adverse events. The present invention automatically processes, integrates, and analyzes text-based sources and generates real time outcomes and predictions with clinical value. The system enables clinicians to i) efficiently search all generated content, ii) use diagnostic and prognostic metrics derived by the system from the content in the therapy and treatment, iii) detect increased risks of adverse events in a patient's progression.

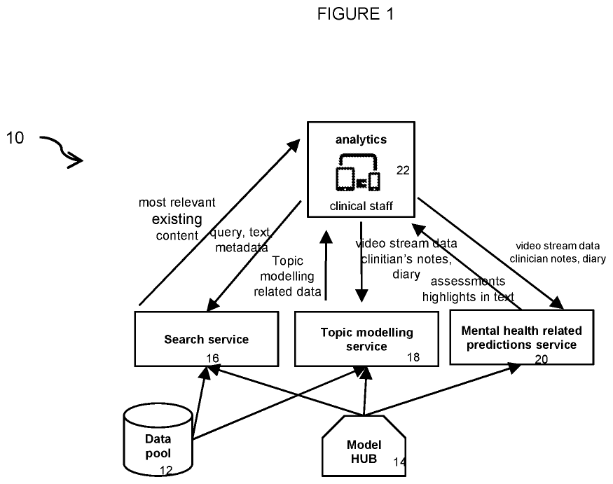

The system is generally shown at 10 in FIG. 1, and includes a data pool 12, model HUB 14, search service 16, topic modeling service 18, mental health related prediction service 20, and analytics 22 all in electronic communication.

The search service 16 uses the data pool 12 content to retreat query and generate the search result. The topic modeling service 18 uses the data pool 12 to define topics for topic extraction.

The model HUB 14 provides retrained models for client facing features and services, such as search service 16, topic modeling service 18, and mental health related predictions service 20.

The search service 16 receives queries, text, and metadata from the analytics 22 and sends back most relevant existing content data pool 12. Search service 16 transforms the query and text metadata from analytics 22 via model version coming from model HUB 14 and uses the new representation of current text to search the data pool 12.

The topic modeling service 18 receives video stream data, for example clinician notes, and diaries from the analytics 22 and sends back topic modeling related data. Topic modeling service 18 searches for the most relevant keywords and phrases describing the topic of the input data. Topic modeling service 18 utilizes the pre-computed data from data pool 12 to execute its function.

The mental health related predictions service 20 receives video stream data, clinician notes, and diaries from the analytics 22 and sends back assessments and highlights in text. The mental health related predictions service 20 selects the right model type or version and makes the prediction for a specific user-facing feature or metric.

The analytics 22 represents user-facing metrics, features and indicators such as visualizations, notifications, lists, counters, charts etc. The analytics 22 takes input from and provides feedback to search service 16, topic modeling service 18 and mental health related prediction service 20.

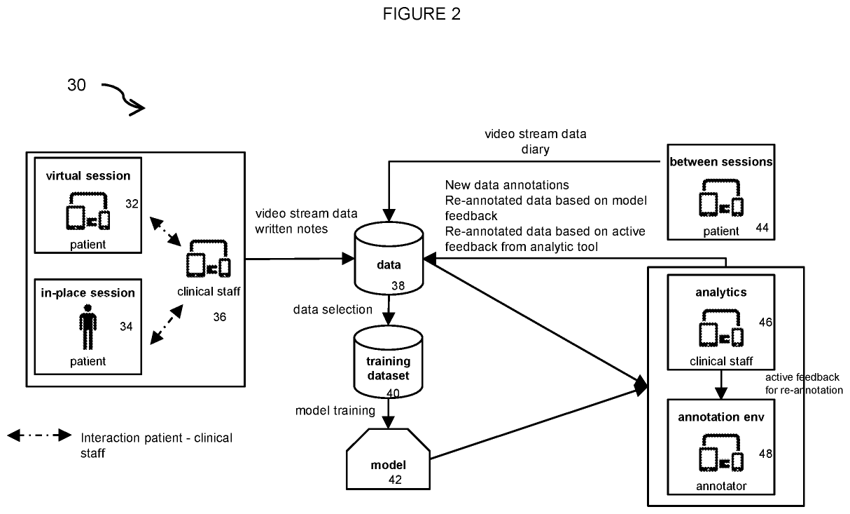

An active learning of the system's models and data collection system is shown in FIG. 2 at 30. While FIG. 1 represents functional architecture of the system in-production version, FIG. 2 describes functional architecture of a sub-system, specifically, architecture of continuous machine learning model training. FIG. 2 sub-system enables the system described in FIG. 1 to improve its diagnostic and prognostic performance over time. The system includes a virtual therapeutic session 32, in-place therapeutic session 34, clinical staff 36, raw data database 38, training dataset 40, trained machine learning model 42, continuous between sessions data collection 44, analytics platform 46, and annotation environment 48.

Clinical staff 36 can receive information from the patient in the virtual therapeutic session 32 or in-place therapeutic session 34.

Video stream data and written notes are sent from the clinical staff database 36 to the internal raw data database 38.

Training data are selected and sent from the internal raw data database 38 to the training dataset database 40. The data 38 also continuously receives video stream data and text-based data (e.g. diaries) from patients in between therapeutic sessions 44.

Model training is performed on the training dataset database 40 and generates a new version of the machine learning model 42.

The analytics platform 46 and annotation environment 48 receive data from internal raw data database 38 and trained machine learning model 42. The analytics platform 46 sends active feedback for re-annotation to the annotation environment 48. The analytics platform 46 and annotation environment 48 send new data annotations, re-annotated data based on model feedback, and re-annotated data based on active feedback from an analytic platform to raw data database 38.

The systems and methods of the present invention can be used with any type of therapy, for any type of disorder of the patient, and in combination with any type of medication.

The sources of the input from the patient can be, but are not limited to, analysis of free-text and structured text responses to diagnostic assignments/tasks, patient's diaries, journals, worry script, transcripts of patient-clinical staff discussions, written/transcribed answers to structured set of questions, and audio recordings.

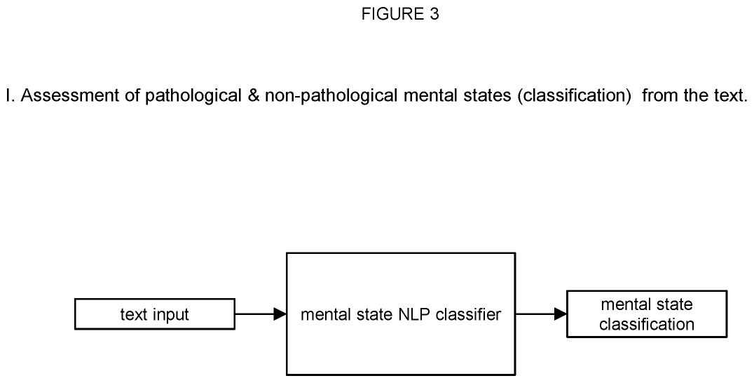

The system 10 can include an assessment of pathological and non-pathological mental states (classification) from patient generated text and audio, shown in FIG. 3. The model input can be a text field (or audio transcript) of random length. The model type is a mental state NLP classifier. The model output is probability distribution across selected mental states related to the text. The training data is the patient's texts and transcripts from therapy sessions, diaries, psychotherapy interventions, with annotations of given mental state (pathological or non-pathological) or bootstrapped public data with high correlations with specific mental states. The user flow is texts and audio generated by a first user (patient) is enabled as an input to the mental state NLP classifier model. The model processes provided text in near-real time and identifies correlates and indications of pathological and nonpathological mental states expressed in the text by its author. Results are presented and visualized in a configurable interface to a second user (clinical staff) and alternatively to the first user (patient). The system's output is used in the diagnostic process, in psychotherapy or for continuous patient monitoring.

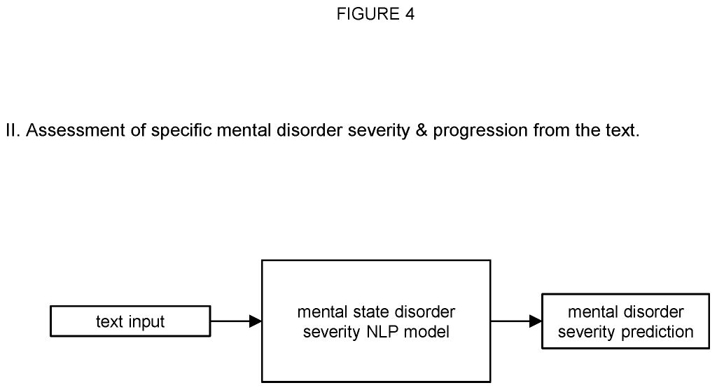

The system 10 can include an assessment of specific mental disorder severity and progression from patient generated text and audio, shown in FIG. 4. The model input is a text field (or audio transcript) of random length. The model type is mental disorder severity NLP classifier. The model output is a score in range 0-100 representing severity of a specific mental disorder and its progression in time. The training data is texts and transcripts from therapy sessions, diaries, psychotherapy interventions, with severity annotations of given mental state (pathological or non-pathological) or bootstrapped public data with high correlations with specific mental states severity. The user flow is texts and audio generated by a first user (patient) that is enabled as an input to the mental disorder severity NLP classifier model. The model processes provided text in near-real time and identifies correlates and indications of severity of a specific mental disorder and/or it's progression, detected in the text. Results are presented and visualized in a configurable interface to a second user (clinical staff) and alternatively, to the first user (patient). The system's output is used in the diagnostic process, in psychotherapy or for continuous patient monitoring.

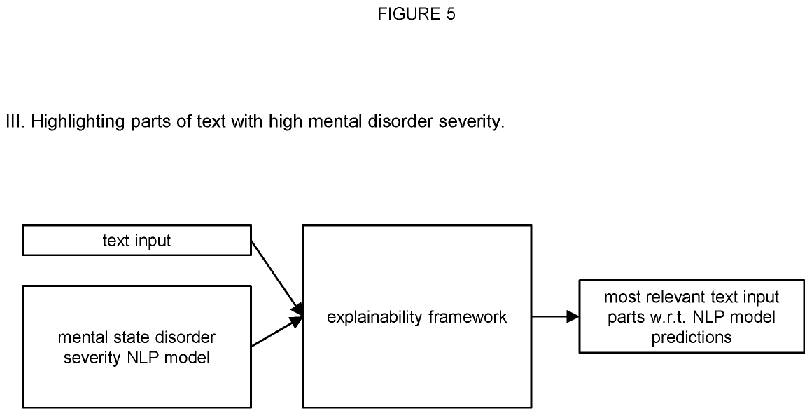

The system 10 can include highlighting parts of text with high mental disorder severity, shown in FIG. 5. The model input is text field (or audio transcript) of random length, output from mental disorder severity text classifier and/or mental state text classifier. The model type is the model's explainability framework. The model output is (for example) color-coded parts of text with high severity. High relevance text extracts. Severity scores per topic. List of n-grams correlated with a specific mental disorder the most. Automatic alerts. The training data is texts and transcripts from therapy sessions, diaries, psychotherapy interventions, with severity annotations of given mental state (pathological or non-pathological) or bootstrapped public data with high correlations with specific mental states severity. The user flow is texts and audio generated by a first user (patient) that is enabled as an input to the explainability framework of the NLP model. The framework plots text in near-real time and visually highlights correlates and indications of severity of a specific mental disorder and/or its progression, detected in the broader body of text. Results are presented and visualized in a configurable interface to the second user (clinical staff) and alternatively, to the first user (patient). The system's 10 output is used in the diagnostic process, in psychotherapy or for continuous patient monitoring.

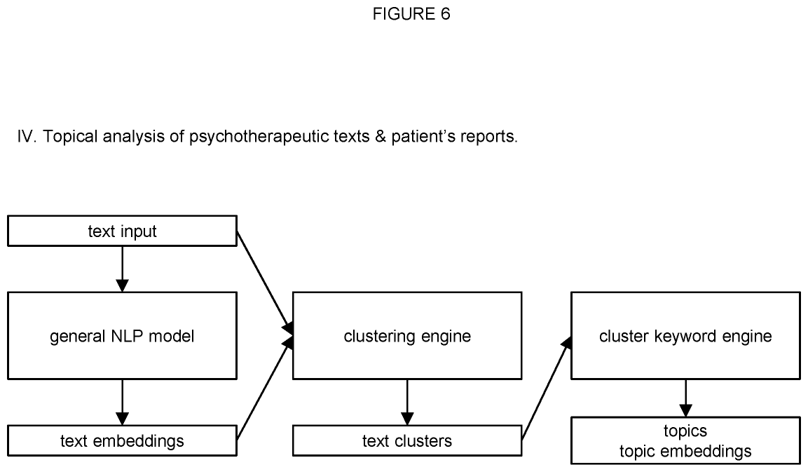

The system 10 can include topical analysis of psychotherapeutic texts, audio transcripts and patient's reports, shown in FIG. 6. The model input is a text field (or audio transcript) of random length. The model type is a general NLP model with following hyperparameters: level of diversity of keywords, number of keywords per topic, minimal relevance score of keywords. The model output is keywords with scores assigned to them representing the level of importance. The training data is models trained on general text corpus. The user flow is texts and audio generated by the first user (patient) is enabled as an input to the general NLP model. The model processes text in near-real time and identifies clinically and therapeutically relevant topics mentioned by the author. These topics are represented by a set of associated keywords. Topics and keyword groups are presented and visualized in a configurable interface to the second user (clinical staff) and alternatively, to the first user (patient). Topics can be ordered by importance or by: specific topic criticality, relevance, frequency and repetition of topics, relation to other topics, relation to diagnosis, relation to disorder progression, relation to therapeutic process or stage, embeddings for all the elements (keywords, topics, paragraphs and other metrics). The system's 10 output is used in the diagnostic process, in psychotherapy or for continuous patient monitoring.

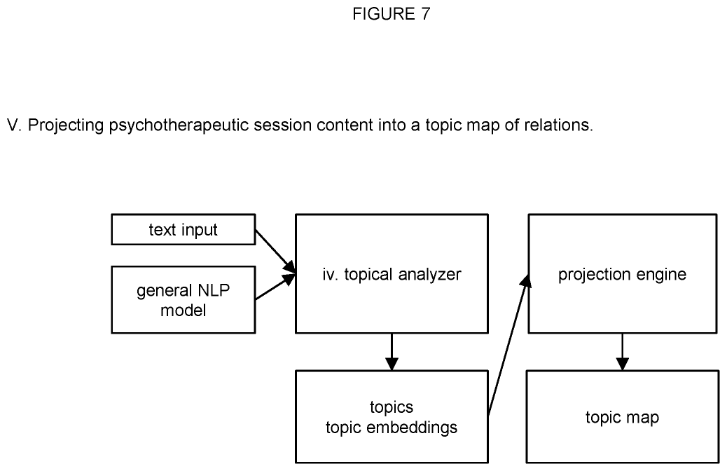

The system 10 can include projecting psychotherapeutic session content into a topic map of relations, shown in FIG. 7. The model input is output of topical analysis of psychotherapeutic texts and patient's reports, with embeddings for all the elements (keywords, topics, paragraphs and other metrics). There can be visual architecture of the plot and associated interactive features. The model output is each text/paragraph of the text visualized into a multi-dimensional topical map where distance represents a semantic similarity and highlighted areas representing distinctive topics. This can be combined with disorder severity and progression indicators to plot trends. Output properties are visual tracking of certain topic development in time on the semantic map (select topic and time range, watch animation), tracking of mental state/severity of topic across time, highlighting critical topics for a specific patient, across therapeutic time range, highlighting semantically related topics with a variable: similarity threshold, highlighting only detected topics associated with a specific pathological or non-pathological mental state, mental disorder severity or type. Model variables are a minimal amount of text belonging to topic to actually form the topic, minimal probability of text belonging to given topic, clustering algorithm variables (e.g. HDBSCAN algorithm properties), n-gram size represents how many vocabulary words can form one text entity. The user flow is texts and audio generated by the first user (patient) is enabled as an input to the visualization framework and plotting architecture. The system processes the text variables in near-real time and identifies correlates and indications of severity of a specific mental disorder and/or its progression for a specific patient or patient group, extracted from the broader body of text. Results are plotted on a multidimensional topical map with configurable properties (see Model output). Interface for the second user (clinical staff) and alternatively, to the first user (patient) enables a visual analysis of extracted and mapped topics and its relation to diagnosis, progression, other topics, and therapeutic intervention. The system's 10 output is used in the diagnostic process, in psychotherapy or for continuous patient monitoring.

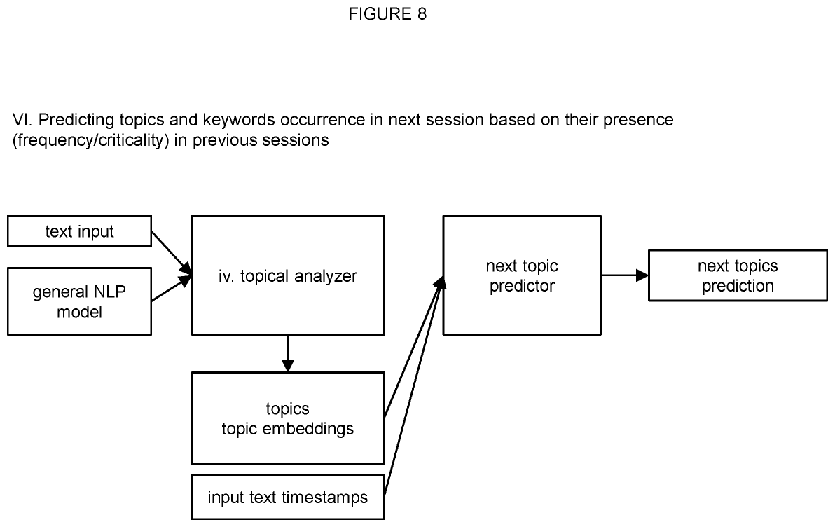

The system 10 includes predicting topics' and keywords' occurrence in the next therapeutic session, based on their presence (frequency and criticality) in previous sessions, shown in FIG. 8. The model input is previous sessions (text field or audio transcript of random length) with timestamps. The model type is general NLP model (to create topics), general model to predict next session topics. The model output is probability distribution over next topics. The user flow is texts and audio generated by the first user (patient) is enabled as an input to the general NLP model and general prediction model. The system processes text in near-real time and identifies topics and keywords most likely to occur in future therapeutic interactions (psychotherapy dialogues, monologues, and future patient generated texts). Results are presented and visualized in a configurable interface to the second user (clinical staff) and alternatively, to the first user (patient). The identified topics and keywords can be presented by the order of their predicted probability to reoccur or by their criticality to therapeutic process (or both as an index). The system's 10 output is used in the diagnostic process, in psychotherapy or for continuous patient monitoring.

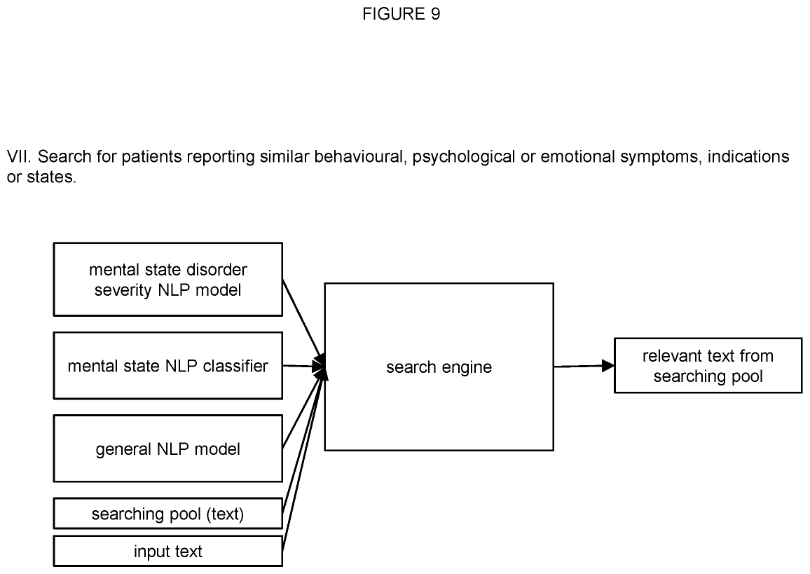

The system 10 can include patient generated text-search for patients reporting similar behavioral, psychological or emotional symptoms, disorder indications or mental states, shown in FIG. 9. The model input is a text field (or audio transcript) of random length. The model type is a general NLP model with following hyperparameters: minimal level of similarity, and distance metric. The model output is pointer to session or patient with similar content/topics. The training data is in the first iteration, a pretrained model is used, further models can be finetuned on the same datasets as for classification of mental health problems. The user flow is texts and audio generated by the first user (patient) is enabled as an input to the general NLP model with hyperparameters of minimal level of similarity and a distance metric. The system processes text in near-real time and identifies commonalities (by keyword co-occurrence, topic co-occurrence and relations between them). Results are i) an arrayed couplets (or groups) of patients, behavior, psychological, emotional indicators reported in processed text, ii) a set of pointers to therapeutic event (e.g. a session), to a patient or patient groups with system-detected similarities in reported content or topics—are presented and visualized in a configurable interface to the second user (clinical staff) and alternatively, to the first user (patient) as text snippets, text extractions or in-text highlights of configurable length. The automatic results hold text with high therapeutic and clinical relevance, grouping entities and events with reported similar clinical indications and experiences (e.g. trauma). The system's 10 output is used in the diagnostic process, in psychotherapy or for continuous patient monitoring.

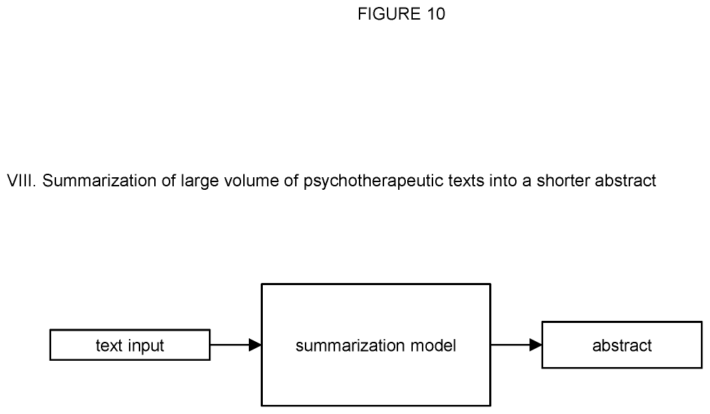

The system 10 can include summarization of a large volume of patient generated texts into a shorter abstract(s) with clinical and diagnostic relevance, shown in FIG. 10. The model input is a patient generated text field (or audio transcript) of random length. The model type is the NLP text summarization model. The model output is an abstract of larger text describing the content in high level, emphasizing the critical aspects of the large psychotherapeutic text input. The training data is a transcript of a session and examples of abstracts and clinical summarizations from a psychotherapeutic session. The user flow is text and audio generated by the first user (patient) is enabled as an input to the NLP text summarization model. The system processes text in near-real time and summarizes the large body of text to compact abstracts, extracting the most relevant moments in the text. Results are presented and visualized in a configurable interface to the second user (clinical staff) and alternatively, to the first user (patient). The automatically generated abstracts hold text with high therapeutic and clinical relevance. The system's 10 output is used in the diagnostic process, in psychotherapy or for continuous patient monitoring.

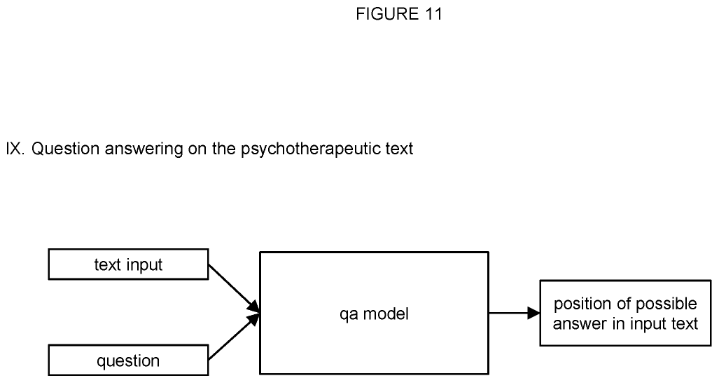

The system 10 can include question answering on the patient generated text, shown in FIG. 11. The model input is the patient's generated pool of texts for use as a source for general question answering, and questions. The model type is a general NLP question answering model. The model output is pointer to the pool of the text(s) where the answer could be. The training data is a dataset of tuples of texts, questions, and pointers to answers. The user flow is texts and audio generated by the first user (patient) is enabled as an input to the general NLP question answering model. The user formulates a question (in audio or text) as an input into an interface configurable by search restrictions for clinical relevance, therapeutic indications, time scale and other types of search restrictions. The system processes text in near-real time. Results are a set of pointers to the locations in the pool of the text(s) where the most likely answer could be—are presented and visualized in a configurable interface to the second user (clinical staff) and alternatively, to the first user (patient) as text snippets and text extractions of configurable length or in-text highlights. The automatic results hold text with high therapeutic and clinical relevance. The system's 10 output is used in the diagnostic process, in psychotherapy or for continuous patient monitoring.

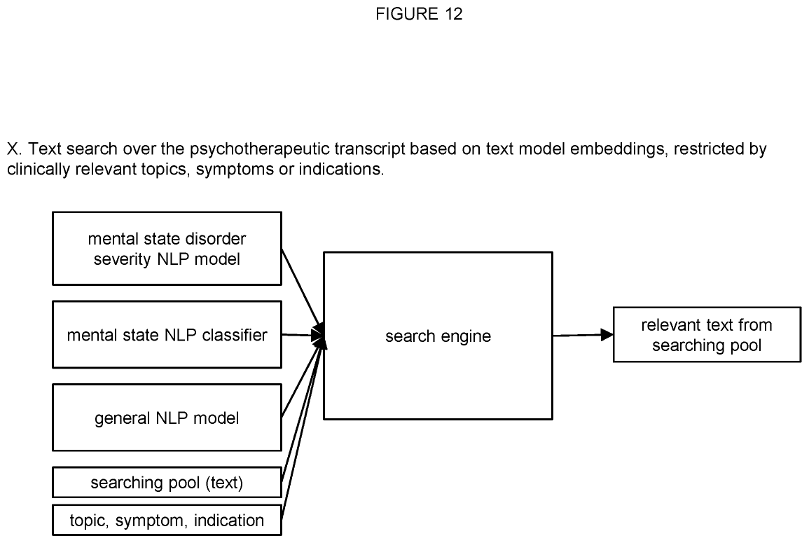

The system 10 can include text search over the patient generated text and transcripts based on text model embeddings, restricted by clinically relevant topics, symptoms, or indications, shown in FIG. 12. The model input is a pool of texts the user wants to search through which can be indexed using paragraph embeddings, query in terms of words or sentences (optional also severity of mental health problems related to query). The model type is a general NLP embedding model. The model output is most semantically similar data found in the text pool, represented by paragraphs, keywords or sentences. The training data is texts and transcripts from therapy sessions, diaries, psychotherapy interventions, with annotations of given mental state (pathological or non-pathological) or bootstrapped public data with high correlations with specific mental states, for embedding finetuning in the next interactions. The user flow is texts and audio generated by the first user (patient) is enabled as an input to the general NLP embedding model. The user inputs a search query into an interface configurable by search restrictions for clinical relevance, therapeutic indications, time scale and other types of search restrictions. The system processes text in near-real time. Results are a list of most semantically similar data found in the text pool—are presented and visualized in a configurable interface to the second user (clinical staff) and alternatively, to the first user (patient) as text snippets and text extractions of configurable length. The automatically generated text snippets hold text with high therapeutic and clinical relevance. The system's 10 output is used in the diagnostic process, in psychotherapy or for continuous patient monitoring.

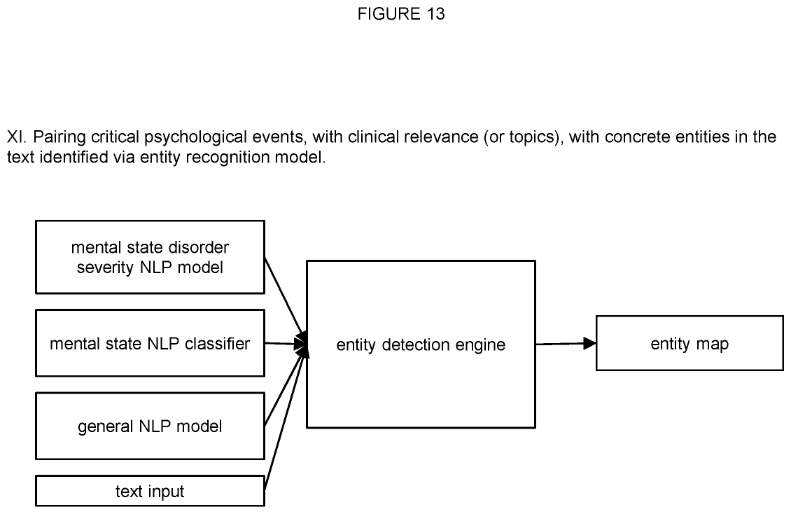

The system 10 can include pairing critical psychological events, with clinical relevance, with concrete entities in the patient generated text identified via entity recognition model, shown in FIG. 13. The model input is a patient generated text field (or audio transcript) of random length. The model type is the NER model, the mental disorder severity NLP classifier and/or the mental state NLP classifier. The model output is entities (people, organizations, locations) mostly related to source of anxiety for given text, their sentiment relations and impact in the text and development of relations in time. The training data is texts and transcripts from therapy sessions, diaries, psychotherapy interventions, with annotations of given entities in text. The user flow is texts and audio generated by the first user (patient) is enabled as an input to the NER model, the mental disorder severity NLP classifier or/and the mental state NLP classifier. The system processes text in near-real time and identifies topics (defined by a set of keywords), events, entities (e.g. people, institutions, organizations, groups, objects etc.). There is clinical relevance or high value for the therapeutic process. Results are presented and visualized in a configurable interface to the second user (clinical staff) and alternatively, to the first user (patient). The identified events, topics and entities and keywords are contextually paired, creating problem space networks represented by nodes (events, entities topics) and edges (their relations with direction and power or quality), or can be presented by the order of their criticality to the therapeutic process. The system's 10 output is used in the diagnostic process, in psychotherapy or for continuous patient monitoring.

The present invention also provides for a method of analyzing a patient, by processing, analyzing, and managing a patient's and clinical staff's text and audio input, and informing and augmenting diagnostic and prognostic processes, identifying improvement and deterioration of a patient's mental state, and identifying adverse events in psychotherapy, counseling, and other mental health management activities. These steps can be performed as described above and include the components described above.

The present invention also provides for a system 10 for processing, analyzing, and managing clinical and diagnostic texts and audio transcripts. The goal of the system is to identify and highlight recurrent topics, critical moments, and clinically valuable moments in clinical staff's feetext notes, to manage content/information load for clinical professionals, and to offer a retrospective view or an overview of a patient's treatment progress (intra and inter session).

The sources of the input from can be, but are not limited to, clinical staff's clinical notes from patient's treatment and therapy, initial assessments notes, progress notes, non-clinical notes from patient's treatment and therapy, drug administration notes, clinical and research staff notes, treatment plans, prescriptions, audio recordings, and release documentation.

The system can include assessment of pathological and non-pathological mental states (classification) from clinical and diagnostic texts and audio. The model input is a text field (or audio transcript) of random length. The model type is a mental state NLP classifier. The model output is probability distribution across selected mental states related to the text. The training data is the patient's texts and transcripts from therapy sessions, diaries, psychotherapy interventions, clinical staff's diagnostic notes from patient's treatment and therapy, non-clinical notes from the patient's treatment and therapy, drug administration notes, clinical and research staff notes, prescriptions, release documentation, with annotations of given mental state (pathological or non-pathological) or bootstrapped public data with high correlations with specific mental states. The user flow is texts and audio generated by the second user (clinical staff) that is enabled as an input to the mental state NLP classifier model. The model processes provided text in near-real time and identifies correlates and indications of pathological and nonpathological mental states expressed in the text by its author. Results are presented and visualized in a configurable interface to the second user (clinical staff) and alternatively to the first user (patient). The system's output is used in the diagnostic process, in psychotherapy or for continuous patient monitoring.

The system can include assessment of specific mental disorder severity and progression from clinical and diagnostic texts and audio. The model input is a clinical and diagnostic text field (or audio transcript) of random length. The model type is mental disorder severity NLP classifier. The model output is score in range 0-100 representing severity of a specific mental disorder and its progression in time. The training data is texts and transcripts from therapy sessions, diaries, psychotherapy interventions, clinical staff's diagnostic notes from patient's treatment and therapy, non-clinical notes from the patient's treatment and therapy, drug administration notes, clinical and research staff notes, prescriptions, release documentation, with severity annotations of given mental state (pathological or non-pathological) or bootstrapped public data with high correlations with specific mental states severity. The user flow is texts and audio generated by the second user (clinical staff) that is enabled as an input to the mental disorder severity NLP classifier model. The model processes provided text in near-real time and identifies correlates and indications of severity of a specific mental disorder and/or its progression, detected in the text. Results are presented and visualized in a configurable interface to the second user (clinical staff) and alternatively, to the first user (patient). The system's output is used in the diagnostic process, in psychotherapy, or for continuous patient monitoring.

The system can include highlighting parts of clinical and diagnostic texts with high mental disorder severity. The model input is text field (or audio transcript) of random length, output from mental disorder severity text classifier and/or mental state text classifier. The model type is the model's explainability framework. The model output is (for example) color coded parts of text with high severity, high relevance text extracts, severity scores per topic, list of n-grams correlated with a specific mental disorder the most, and automatic alerts. The training data is texts and transcripts from therapy sessions, diaries, psychotherapy interventions, clinical staff's diagnostic notes from patient's treatment and therapy, non-clinical notes from patient's treatment and therapy, drug administration notes, clinical and research staff notes, prescriptions, release documentation, with severity annotations of given mental state (pathological or non-pathological) or bootstrapped public data with high correlations with specific mental states severity. The user flow is texts and audio generated by the second user (clinical staff) that is enabled as an input to the explainability framework of the NLP model. The framework plots text in near-real time and visually highlights correlates and indications of severity of a specific mental disorder and/or it's progression, detected in the broader body of text. Results are presented and visualized in a configurable interface to the second user (clinical staff) and alternatively, to the first user (patient). The system's output is used in the diagnostic process, in psychotherapy, or for continuous patient monitoring.

The system can include topical analysis of clinical and diagnostic texts, audio transcripts, and clinical staff notes and reports. The model input is a text field (or audio transcript) of random length. The model type is a general NLP model with the following hyperparameters: level of diversity of keywords, number of keywords per topic, and minimal relevance score of keywords. The model output is keywords with a score assigned to them representing the level of importance. The training data is models trained on general text corpus. The user flow is texts and audio generated by the second user (clinical staff) that is enabled as an input to the general NLP model. The model processes text in near-real time and identifies clinically and therapeutically relevant topics mentioned by the author. These topics are represented by a set of associated keywords. Topics and keyword groups are presented and visualized in a configurable interface to the second user (clinical staff) and alternatively, to the first user (patient). Topics can be ordered by importance or by: specific topic criticality, relevance, frequency and repetition of topics, relation to other topics, relation to diagnosis, relation to disorder progression, relation to therapeutic process or stage, embeddings for all the elements (keywords, topics, paragraphs and other metrics). The system's output is used in the diagnostic process, in psychotherapy or for continuous patient monitoring.

The system can include projecting clinical and diagnostic texts and audio generated at psychotherapeutic sessions into a topic map of relations. The model input is output of topical analysis of psychotherapeutic texts and patient's reports, with embeddings for all the elements (keywords, topics, paragraphs and other metrics). There can be visual architecture of the plot and associated interactive features. The model output is each text/paragraph of the text visualized into a multi-dimensional topical map where distance represents a semantic similarity and highlighted areas representing distinctive topics. This can be combined with disorder severity and progression indicators to plot trends. The output properties are visual tracking of certain topic development in time on the semantic map (select topic and time range, watch animation), tracking of mental state/severity of topic across time, highlighting critical topics for a specific patient, across therapeutic time range, highlighting semantically related topics with a variable: similarity threshold, and highlighting only detected topics associated with a specific pathological or non-pathological mental state, mental disorder severity or type. The model variables are a minimal amount of text belonging to topic to actually form the topic, minimal probability of text belonging to given topic, clustering algorithm variables (e.g. HDBSCAN algorithm properties), and n-gram size represents how many vocabulary words can form one text entity. The user flow is texts and audio generated by the second user (clinical staff) that is enabled as an input to the visualization framework and plotting architecture. The system processes the text variables in near-real time and identifies correlates and indications of severity of a specific mental disorder and/or its progression for a specific patient or patient group, extracted from the broader body of text. Results are plotted on a multidimensional topical map with configurable properties (see Model output). An interface for the second user (clinical staff) and alternatively, to the first user (patient) enables a visual analysis of extracted and mapped topics and its relation to diagnosis, progression, other topics, or a specific past therapeutic intervention. The system's output is used in the diagnostic process, in psychotherapy or for continuous patient monitoring.

The system can include predicting topics' and keywords' occurrence in the next therapeutic session, based on their presence (frequency and criticality) in previous sessions. The model input is previous sessions (text field or audio transcript of random length) with timestamps, psychotherapy interventions, clinical staff's diagnostic notes from the patient's treatment and therapy, non-clinical notes from the patient's treatment and therapy, drug administration notes, clinical and research staff notes, prescriptions, and release documentation. The model type is general NLP model (to create topics), general model to predict next session topics. The model output is probability distribution over next topics. The user flow is texts and audio generated by the second user (clinical staff) that is enabled as an input to the general NLP model and general prediction model. The system processes text in near-real time and identifies topics and keywords most likely to occur in future therapeutic interactions (psychotherapy dialogues, monologues, and future patient generated texts). Results are presented and visualized in a configurable interface to the second user (clinical staff) and alternatively, to the first user (patient). The identified topics and keywords can be presented by the order of their predicted probability to reoccur or by their criticality to therapeutic process (or both as an index). The system's output is used in the diagnostic process, in psychotherapy or for continuous patient monitoring.

The system can include clinical and diagnostic text-search for patients reporting similar behavioral, psychological or emotional symptoms, disorder indications or mental states. The model input is a text field (or audio transcript) of random length. The model type is a general NLP model with following hyperparameters: minimal level of similarity, and distance metric. The model output is pointer to session or patient with similar content/topics. The training data is in the first iteration a pretrained model is used, further models can be finetuned on the same datasets as for classification of mental health disorders. The user flow is texts and audio generated by the second user (clinical staff) that is enabled as an input to the general NLP model with hyperparameters of minimal level of similarity and a distance metric. The system processes text in near-real time and identifies commonalities (by keyword co-occurrence, topic co-occurrence and relations between them). Results are i) an arrayed couplets (or groups) of patients, behavior, psychological, emotional indicators reported in processed text, ii) a set of pointers to therapeutic event (e.g. a session), to a patient or patient groups with system-detected similarities in reported content or topics—are presented and visualized in a configurable interface to the second user (clinical staff) and alternatively, to the first user (patient) as text snippets, text extractions or in-text highlights of configurable length. The automatic results hold text with high therapeutic and clinical relevance, grouping entities and events with reported similar clinical indications and experiences (e.g. trauma). The system's output is used in the diagnostic process, in psychotherapy or for continuous patient monitoring.

The system can include summarization of a large volume of clinical and diagnostic texts into a shorter abstract(s) with clinical and diagnostic relevance. The model input is a patient generated text field (or audio transcript) of random length. The model type is the NLP text summarization model. The model output is an abstract of larger text describing the content in high level, emphasizing the critical aspects of the large psychotherapeutic text input. The training data is transcript of a therapy session and examples of abstracts and clinical summarizations from a psychotherapeutic session, psychotherapy interventions, clinical staff's diagnostic notes from patient's treatment and therapy, non-clinical notes from patient's treatment and therapy, drug administration notes, clinical and research staff notes, prescriptions, and release documentation. The user flow is texts and audio generated by the second user (clinical staff) is enabled as an input to the NLP text summarization model. The system processes text in near-real time and summarizes the large body of text to compact abstracts, extracting the most relevant moments in the text. Results are presented and visualized in a configurable interface to the second user (clinical staff) and alternatively, to the first user (patient). The automatically generated abstracts hold text with high therapeutic and clinical relevance. The system's output is used in the diagnostic process, in psychotherapy or for continuous patient monitoring.

The system can include question answering on the clinical and diagnostic texts. The model input is the patient's generated pool of texts for use as source for general question answering, and question. The model type is a general NLP question answering model. The model output is pointer to the pool of the text(s) where the answer could be. The training data is a dataset of tuples of texts, questions, and pointers to answers. The user flow is texts and audio generated by the second user (clinical staff) that is enabled as an input to the general NLP question answering model. The user formulates a question (in audio or text) as an input into an interface configurable by search restrictions for clinical relevance, therapeutic indications, time scale and other types of search restrictions. The system processes text in near-real time. Results are a set of pointers to the locations in the pool of the text(s) where the most likely answer could be that are presented and visualized in a configurable interface to the second user (clinical staff) and alternatively, to the first user (patient) as text snippets and text extractions of configurable length or in-text highlights. The automatic results hold text with high therapeutic and clinical relevance. The system's output is used in the diagnostic process, in psychotherapy or for continuous patient monitoring.

The system can include text search over the clinical and diagnostic texts and transcripts based on text model embeddings, restricted by clinically relevant topics, symptoms or indications. The model input is a pool of texts the user wants to search through which is indexed using paragraph embeddings, query in terms of words or sentences (optional also severity of mental health problems related to query). The model type is a general NLP embedding model. The model output is most semantically similar data found in the text pool, represented by paragraphs, keywords, or sentences. The training data is texts and transcripts from therapy sessions, diaries, psychotherapy interventions, clinical staff's diagnostic notes from patient's treatment and therapy, non-clinical notes from patient's treatment and therapy, drug administration notes, clinical and research staff notes, prescriptions, release documentation or bootstrapped public data with high correlations with specific mental states, for embedding finetuning in the next interactions. The user flow is texts and audio generated by the second user (clinical staff) is enabled as an input to the general NLP embedding model. The user inputs a search query into an interface configurable by search restrictions for clinical relevance, therapeutic indications, time scale and other types of search restrictions. The system processes text in near-real time. Results are a list of most semantically similar data found in the text pool that are presented and visualized in a configurable interface to the second user (clinical staff) and alternatively, to the first user (patient) as text snippets and text extractions of configurable length. The automatically generated text snippets hold text with high therapeutic and clinical relevance. The system's output is used in the diagnostic process, in psychotherapy or for continuous patient monitoring.

The system can include pairing critical psychological events, with clinical relevance, with concrete entities in the clinical and diagnostic texts, identified via entity recognition model. The model input is a patient generated text field (or audio transcript) of random length. The model type is the NER model, the mental disorder severity NLP classifier and/or the mental state NLP classifier. The model output is entities (people, organizations, locations) mostly related to source of anxiety for given text, their sentiment relations and impact in the text, and development of relations in time. The training data is texts and transcripts from therapy sessions, psychotherapy interventions, clinical staff's diagnostic notes from patient's treatment and therapy, non-clinical notes from patient's treatment and therapy, drug administration notes, clinical and research staff notes, prescriptions, release documentation. The user flow is texts and audio generated by the second user (clinical staff) is enabled as an input to the NER model, the mental disorder severity NLP classifier or/and the mental state NLP classifier. The system processes text in near-real time and identifies: topics (defined by a set of keywords), events, entities (e.g. people, institutions, organizations, groups, objects etc.) with clinical relevance or high value for the therapeutic process. Results are presented and visualized in a configurable interface to the second user (clinical staff) and alternatively, to the first user (patient). The identified events, topics and entities and keywords are contextually paired, creating problem space networks represented by nodes (events, entities topics) and edges (their relations with direction and power or quality) or can be presented by the order of their criticality to the therapeutic process. The system's output is used in the diagnostic process, in psychotherapy or for continuous patient monitoring.

The present invention provides for a method of analyzing clinical and diagnostic texts, by processing, analyzing, and managing clinical and diagnostic texts, identifying and highlighting recurrent topics, critical moments, and clinically valuable moments in clinical staff's feetext notes, managing content/information load for clinical professionals, and offering a retrospective view or an overview of a patient's treatment progress. These steps can be performed as described above and include the components described above.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

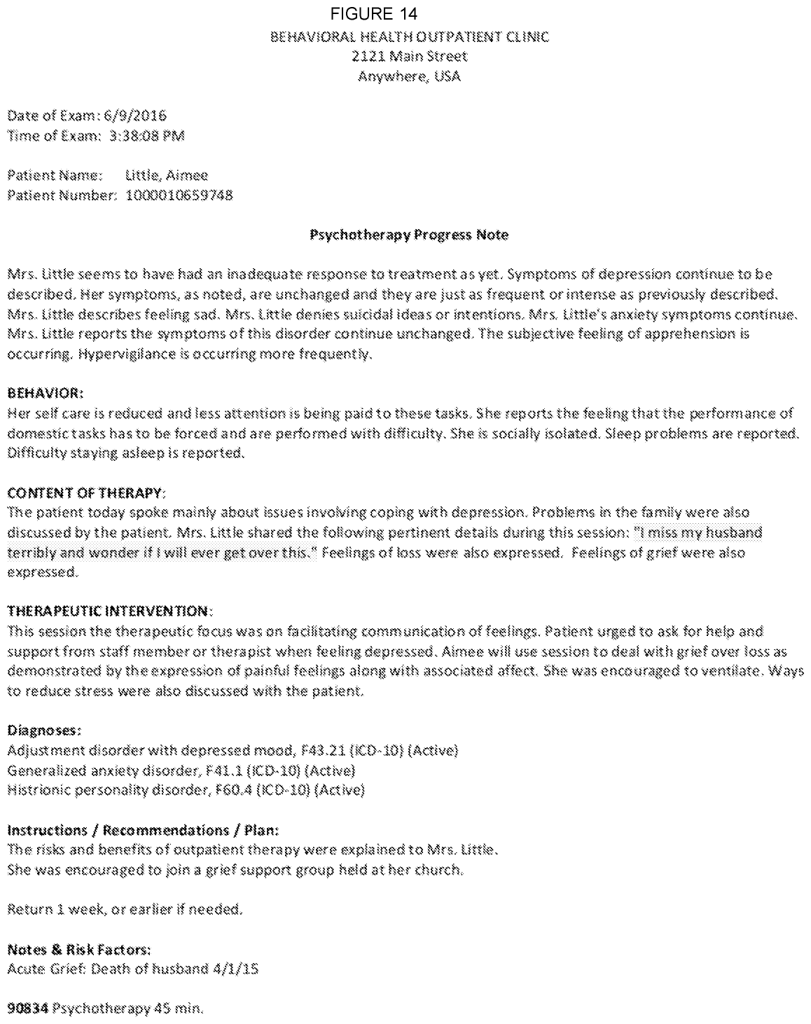

FIG. 14 shows an example of individual therapy notes. This is a general example of a machine learning model input, i.e. a text with potential clinical value that is processed by the system 10.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible considering the above teachings. It is, therefore, to be understood that within the scope of the appended claims, the invention can be practiced otherwise than as specifically described.

What is claimed is:

1. A method of analyzing a patient's mental state, including the steps of:
automatically processing, integrating, and analyzing text-based and audio-based sources from the patient and clinical staff; and
generating real-time outcomes and predictions.

2. The method of claim 1, wherein the sources are chosen from the group consisting of analysis of free-text and structured text responses to diagnostic assignments/tasks, patient's diaries, journals, worry script, transcripts of patient-clinical staff discussions, written/transcribed answers to structured set of questions, and audio recordings.

3. The method of claim 1, further providing an assessment of pathological and non-pathological mental states from patient generated text and audio.

4. The method of claim 1, wherein said generating step further includes presenting results in a visual interface for a user.

5. The method of claim 1, further providing an assessment of specific mental disorder severity and progression from patient generated text and audio.

6. The method of claim 1, further including the step of highlighting parts of text with high mental disorder severity.

7. The method of claim 1, further including the step of providing topical analysis of psychotherapeutic texts, audio transcripts, and patient's reports.

8. The method of claim 1, further including the step of projecting psychotherapeutic session content into a topic map of relations.

9. The method of claim 1, further including the step of predicting topics' and keywords' occurrence in a next therapeutic session, based on their presence in previous sessions.

10. The method of claim 1, further providing patient generated text-search for patients reporting similar behavioral, psychological or emotional symptoms, disorder indications or mental states.

11. The method of claim 1, further providing the step of summarizing of a volume of patient generated texts into a shorter abstracts with clinical and diagnostic relevance.

12. The method of claim 1, further including the step of question answering on the patient generated text.

13. The method of claim 1, further including the step of text searching over the patient generated text and transcripts based on text model embeddings, restricted by clinically relevant topics, symptoms, or indications.

14. The method of claim 1, further including the step of pairing critical psychological events with concrete entities in the patient generated text identified via entity recognition model.

15. A system for processing, analyzing, and managing a patient's input comprising a data pool, model HUB, search service, topic modeling service, mental health related prediction service, and analytics all in electronic communication.

16. The system of claim 15, wherein said search service uses said data pool content to retreat query and generate a search result.

17. The system of claim 15, wherein said topic modeling service uses said data pool to define topics for topic extraction.

18. The system of claim 17, wherein said topic modeling service receives video stream data and diaries from said analytics and sends back topic modeling related data.

19. The system of claim 15, wherein said model HUB provides retrained models for client facing features and services.

20. The system of claim 15, wherein said search service receives queries, text, and metadata from said analytics and sends back most relevant existing content data pool, and transforms query and text metadata from analytics via model version coming from said model HUB and uses a new representation of current text to search said data pool.

21. The system of claim 15, wherein said mental health related prediction service receives video stream data, clinician notes, and diaries from said analytics and sends back assessments and highlights in text.

22. The system of claim 15, wherein said analytics includes user-facing metrics including visualizations, notifications, lists, counters, and charts and receives input from and provides feedback to said search service, said topic modeling service, and said mental health related prediction service.

23. A method of analyzing a patient, including the steps of:
processing, analyzing, and managing a patient's and clinical staff's text and audio input;
informing and augmenting diagnostic and prognostic processes, identifying improvement and deterioration of a patient's mental state; and
identifying adverse events in psychotherapy, counseling, and mental health management activities.

24. A system for processing, analyzing, and managing clinical and diagnostic texts and audio transcripts comprising a data pool, model HUB, search service, topic modeling service, mental health related prediction service, and analytics all in electronic communication, wherein said system is able to identify and highlight recurrent topics, critical moments, and clinically valuable moments in clinical staff's feetext notes, to manage content/information load for clinical professionals, and to offer a retrospective view or an overview of a patient's treatment progress.

25. A method of analyzing clinical and diagnostic texts, including the steps of:
processing, analyzing, and managing clinical and diagnostic texts;
identifying and highlighting recurrent topics, critical moments, and clinically valuable moments in clinical staff's feetext notes;
managing content/information load for clinical professionals; and
offering a retrospective view or an overview of a patient's treatment progress.

26. The method of claim 25, wherein sources of input are chosen from the group consisting of clinical staff's clinical notes from patient's treatment and therapy, initial assessments notes, progress notes, non-clinical notes from patient's treatment and therapy, drug administration notes, clinical and research staff notes, treatment plans, prescriptions, audio recordings, release documentation, and combinations thereof.

27. The method of claim 25, further including the step of assessing pathological and non-pathological mental states from clinical and diagnostic texts and audio.

28. The method of claim 25, further including the step of assessing specific mental disorder severity and progression from clinical and diagnostic texts and audio.

29. The method of claim 25, further including the step of highlighting parts of clinical and diagnostic texts with high mental disorder severity.

30. The method of claim 25, further including the step of providing topical analysis of clinical and diagnostic texts, audio transcripts, and clinical staff notes and reports.

31. The method of claim 25, further including the step of projecting clinical and diagnostic texts and audio generated at psychotherapeutic sessions into a topic map of relations.

32. The method of claim 25, further including the step of predicting topics' and keywords' occurrence in the next therapeutic session, based on their presence in previous sessions.

33. The method of claim 25, further including the step of providing clinical and diagnostic text-search for patients reporting similar behavioral, psychological or emotional symptoms, disorder indications or mental states.

34. The method of claim 25, further including the step of summarizing of a volume of clinical and diagnostic texts into a shorter abstracts with clinical and diagnostic relevance.

35. The method of claim 25, further including the step of question answering on the clinical and diagnostic texts.

36. The method of claim 25, further including the step of text searching over the clinical and diagnostic text and transcripts based on text model embeddings, restricted by clinically relevant topics, symptoms, or indications.

37. The method of claim 25, further including the step of pairing critical psychological events with concrete entities in the clinical and diagnostic texts identified via entity recognition model.

38. The method of claim 25, wherein said generating step further includes presenting results in a visual interface for a user.
